(19) United States
(12) Patent Application Publication
(10) Pub. No.: US 2016/0051274 A1
(43) Pub. Date: Feb. 25, 2016

(54) ROLLING JOINT JAW MECHANISM

(71) Applicant: BRIGHAM YOUNG UNIVERSITY, Provo, UT (US)

(72) Inventors: Larry L. HOWELL, Orem, UT (US); Spencer P. MAGLEBY, Provo, UT (US); Brian D. JENSEN, Orem, UT (US); John Ryan STEGER, Sunnyvale, CA (US); Jordan TANNER, Provo, UT (US); Clayton GRAMES, Provo, UT (US)

(21) Appl. No.: 14/831,699

(22) Filed: Aug. 20, 2015

Related U.S. Application Data
(60) Provisional application No. 62/039,805, filed on Aug. 20, 2014.

(51) Int. Cl. A61B 17/29; B25J 17/02; A61B 19/00; B25J 15/00

(52) U.S. Cl. CPC A61B 17/29; B25J 15/0028; B25J 17/02; A61B 19/2203; Y10S 901/41; Y10S 901/31; A61B 2017/2939; A61B 2017/2947; A61B 2019/2234

(57) ABSTRACT

According to an aspect, a device may include a shaft, a tool portion including a first arm and a second arm, and a split rolling joint including a first curved portion and a second curved portion. The second curved portion may be coupled to the shaft. The first curved portion may include a first split portion and a second split portion. The first split portion may be coupled to the first arm. The second split portion may be coupled to the second arm. At least one of the first split portion and the second split portion may be configured to roll with respect to the second curved portion such that at least one of the first arm and the second arm can move towards or away from each other.

### CROSS-REFERENCE TO RELATED APPLICATION

This application is a Non-provisional of, and claims priority to, U.S. Patent Application No. 62/039,805, filed on Aug. 20, 2014, entitled “Rolling Joint Jaw Mechanism”, which is incorporated by reference herein in its entirety.

### TECHNICAL FIELD

This disclosure relates generally to devices having a split rolling joint coupled to a tool portion having at least two degrees of freedom, and particularly surgical devices having the split rolling element joint and the tool portion with at least two degrees of freedom.

### BACKGROUND

A conventional compliant rolling-element (CORE) joint may include joining two half cylinders with flexures. However, surgical instruments having a conventional CORE joint may succumb to friction, wear, and/or undesirable motion, and may be difficult to use within smaller surgical instruments such as instruments used in laparoscopic and robotic surgical operations.

### SUMMARY

According to an aspect, a device may include a shaft, a tool portion including a first arm and a second arm, and a split rolling joint including a first curved portion and a second curved portion. The second curved portion may be coupled to the shaft. The first curved portion may include a first split portion and a second split portion. The first split portion may be coupled to the first arm. The second split portion may be coupled to the second arm. At least one of the first split portion and the second split portion may be configured to roll with respect to the second curved portion such that at least one of the first arm and the second arm can move towards or away from each other.

In some examples, the device may include one or more of the below features (or any combination thereof). The first split portion and the second split portion may be configured to independently roll with respect to the second curved portion. Each of the first curved portion and the second curved portion may include half a cylinder divided lengthwise. Each of the first split portion and the second split portion may include a curved surface configured to engage with a surface of the second curved portion. Each of the second curved portion, the first split portion, and the second split portion may include a gear surface area. The gear surface area may include a plurality of recesses and protrusions. The gear surface area of the first split portion may be rollably engaged with a first portion of the gear surface area of the second curved portion, and the gear surface area of the second split portion may be rollably engaged with a second portion of the gear surface area of the second curved portion. Each of the second curved portion, the first split portion, and the second split portion may include at least one non-geared surface area. The at least one non-geared surface area may be devoid of gears. The device may include an actuation mechanism configured to control movement of at least one of the first split portion and the second split portion. The shaft may have a diameter between 1 millimeters and 5 millimeters. The tool portion may be a cutter or a grasper.

According to an aspect, a device may include a shaft, a tool portion including a first movable arm and a second movable arm, and a split rolling joint including a first curved portion and a second curved portion. The second curved portion may be coupled to the shaft. The first curved portion may include a first split portion and a second split portion. The first split portion may be coupled to the first movable arm. The second split portion may be coupled to the second movable arm. The first split portion and the second split portion may be configured to independently roll with respect to the second curved portion such that the first movable arm and the second movable arm can move towards or away from each other and can position the tool portion in more than one direction by moving the first and second movable arms as a unit.

In some examples, the device may include one or more of the above and/or below features (or any combination thereof). The second split portion may be disposed adjacent to the first split portion. The second curved portion may include a first row of a gear profile and a second row of the gear profile. The second row may be adjacent to the first row. The gear profile may include a plurality of recesses and protrusions. The first split portion may include a third row of the gear profile. The second split portion may include a fourth row of the gear profile. The third row of the gear profile on the first split portion may be rollably engaged with the first row of the gear profile on the second curved portion. The fourth row of the gear profile on the second split portion may be rollably engaged with the second row of the gear profile on the second curved portion. The device may include a first actuator member coupled to the first movable arm, and a second actuator member coupled to the second movable arm. The movement of the first actuator member may be configured to rotate the first split portion about the second curved portion to move the first movable arm, and the movement of the second actuator member may be configured to rotate the second split portion about the second curved portion to move the second movable arm. At least a portion of the first actuator member may extend along a longitudinal axis of the shaft, and at least a portion of the second actuator member may extend along the longitudinal axis of the shaft. The second curved portion may include a guide configured to guide rotation movement of at least one of the first split portion and the second split portion with respect to the second curved portion. Each of the second curved portion, the first split portion, and the second split portion may include a gear surface area defining a gear profile and at least one non-geared surface area. The at least one non-geared surface area may be devoid of gears.

According to an aspect, a medical device may include a shaft, a tool portion including a first movable arm and a second movable arm, and a split rolling joint including a first curved portion and a second curved portion, where the second curved portion is coupled to the shaft, and the first curved portion includes a first split portion and a second split portion. The first split portion may be coupled to the first movable arm. The second split portion may be coupled to the second movable arm. The second split portion and the second split portion may be configured to independently move with respect to the second curved portion. The first movable arm and the second movable arm may be configured to move independently from each other such that movement of the first split portion and the second split portion provides two degrees of movement.

In some examples, the device may include one or more of the above and/or below features (or any combination thereof). The two degrees of movement may include movement associated with the first split portion rolling on the second curved portion and movement associated with the second split portion rolling on the second curved portion. The two degrees of movement may include movement associated with moving the first movable arm and the second movable arm towards or away from each other and movement associated with positioning the tool portion in more than one direction by rotating the first and second movable arms as a unit.

### BRIEF DESCRIPTION OF THE DRAWINGS

FIG. 1 illustrates a device having a split rolling joint coupled to a shaft and coupled to first and second movable arms of a tool portion according to an aspect.

FIG. 2 illustrates a perspective of the split rolling joint and the first and second movable arms according to an aspect.

FIG. 3A illustrates a perspective of the split rolling joint depicting a gear profile on the curved surfaces of the split rolling joint according to an aspect.

FIG. 3B illustrates a perspective of the split rolling joint of FIG. 3A but with an optional guide according to an aspect.

FIG. 4 illustrates a device depicting an actuation mechanism for controlling the two-degree-of-freedom tool portion according to an aspect.

FIG. 5A illustrates a device according to an aspect.

FIG. 5B illustrates a device according to another aspect.

FIG. 5C illustrates a device according to another aspect.

FIG. 6A illustrates a device according to another aspect.

FIG. 6B illustrates the device of FIG. 6A according to an aspect.

FIG. 6C illustrates the device of FIG. 6A according to an aspect.

FIG. 7A illustrates a device according to another aspect.

FIG. 7B illustrates the device of FIG. 7A according to another aspect.

FIG. 7C illustrates the device of FIG. 7A according to another aspect.

FIG. 8 illustrates geometry and parameters used in deriving equations of motion and force output for the split rolling joint according to an aspect.

FIG. 9 illustrates a graph depicting the required input force for a range of the split rolling joint according to an aspect.

FIG. 10 illustrates a graph of the mechanical advantage of the split rolling joint according to an aspect.

FIG. 11 illustrates the geometry depicting the parameters used to determine the stress states cause by the contact between the upper and lower segments of the split rolling joint according to an aspect.

FIG. 12 illustrates a graph plotting the stress states at the contact point of the split rolling joint according to an aspect.

FIG. 13 illustrates a device having the split rolling joint according to an aspect.

FIG. 14 illustrates a device having the split rolling joint according to another aspect.

FIG. 15 illustrates a device having the split rolling joint according to another aspect.

FIG. 16 illustrates a device having the split rolling joint according to another aspect.

FIG. 17A illustrates the first and second movable arms according to an aspect.

FIG. 17B illustrates a collar component of the split rolling joint according to an aspect.

FIG. 17C illustrates a base component of the split rolling joint according to an aspect.

### DETAILED DESCRIPTION

The terms proximal and distal described in relation to various devices and components are referred with a point of reference. The point of reference may be an operator. The operator may be a person such as a surgeon, a physician, a nurse, a doctor, or a technician who may perform the procedure and operate the medical device as described in this disclosure, or the operator may be a teleoperated or robotic manipulator technology that operates the medical device. The term proximal may refer to an area or portion that is closer or closest to the operator during a surgical procedure. The term distal may refer to an area or portion that is farther or farthest from the operator.

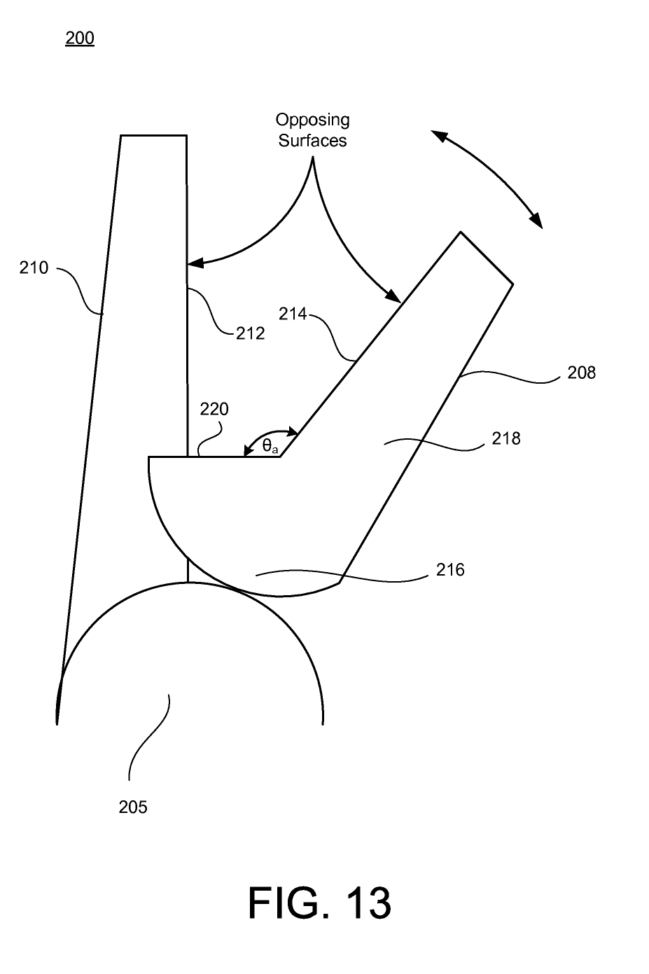

The devices described herein have advantages over some rolling element joints that may be used in a wide variety of grasping, cutting, and manipulating operations. In some examples, a rolling element joint may allow control of an angle of a tool with respect to a mounting shaft. The rolling element joint may be placed at the end of the shaft, before the tool (e.g., cutter or grasper) to improve the dexterity of the tool. In some examples, the rolling element joint may be a Compliant Rolling-Contact Element (CORE) joint that includes two half cylinders. The embodiments described herein include joints having a tool (e.g., gripper) with two degrees of freedom that minimizes or reduces friction. The embodiments described herein have mechanisms providing relatively low friction at small scales. The embodiments described herein include at least a two-degree-of-freedom tool at small scales that involves a minimum or reduced number of parts. In other examples, the embodiments include a tool with one degree-of-freedom (e.g., as explained with reference to FIG. 13).

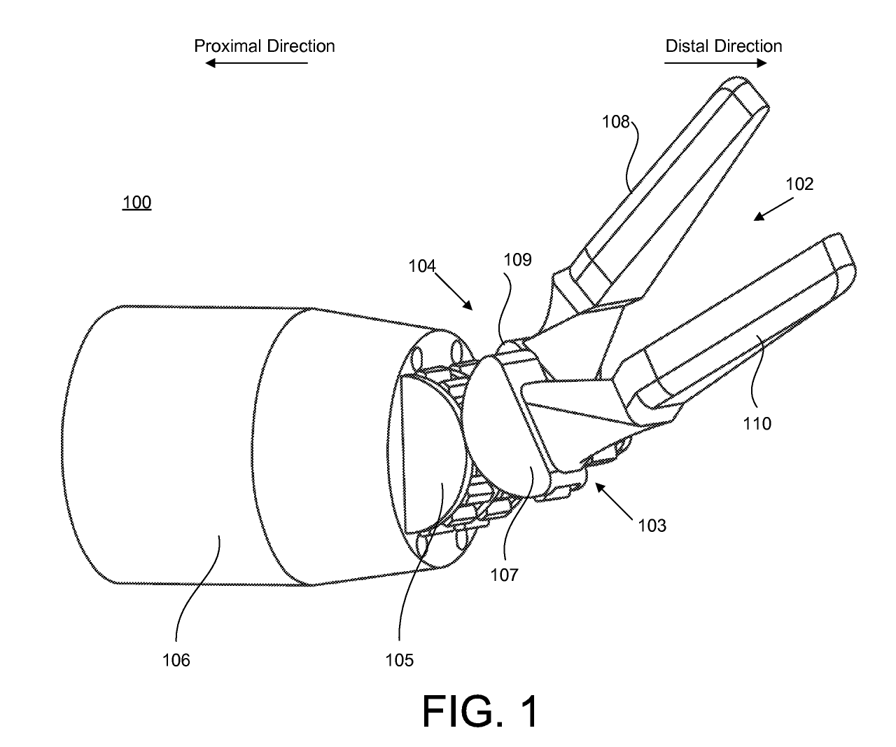

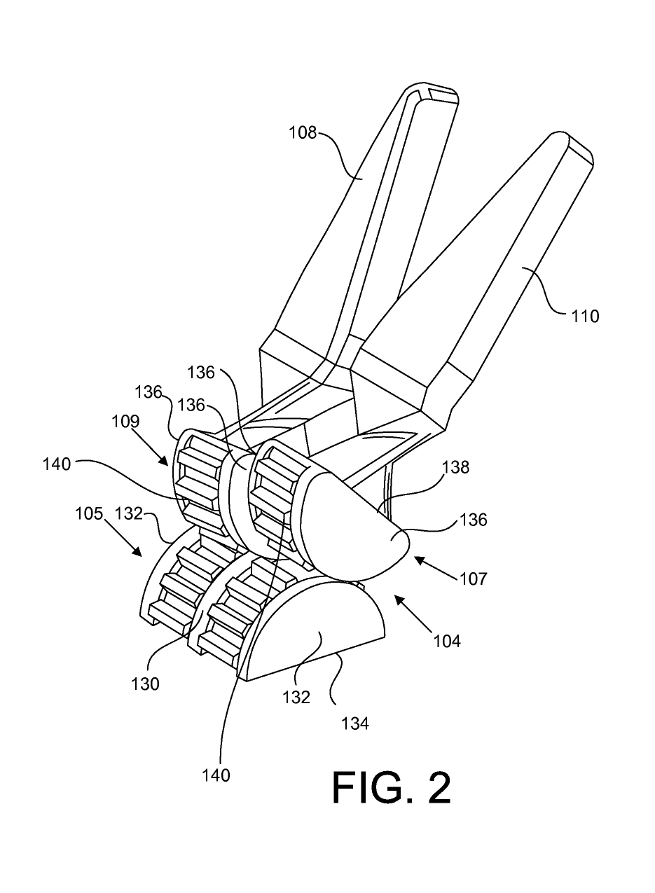

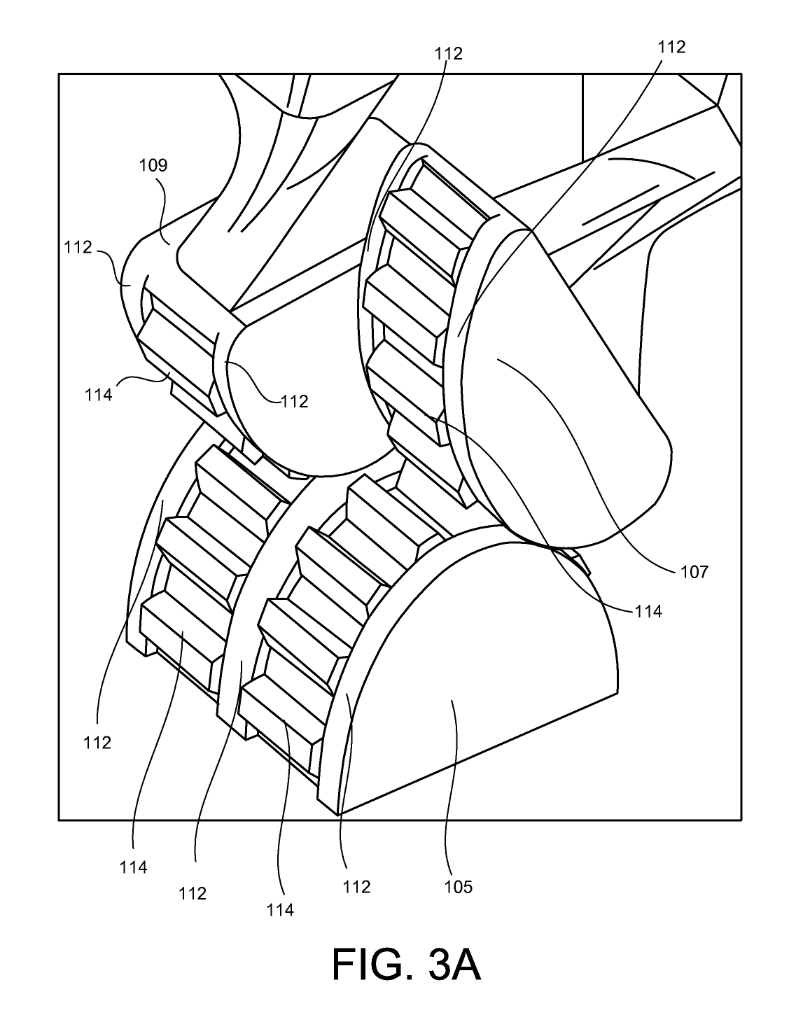

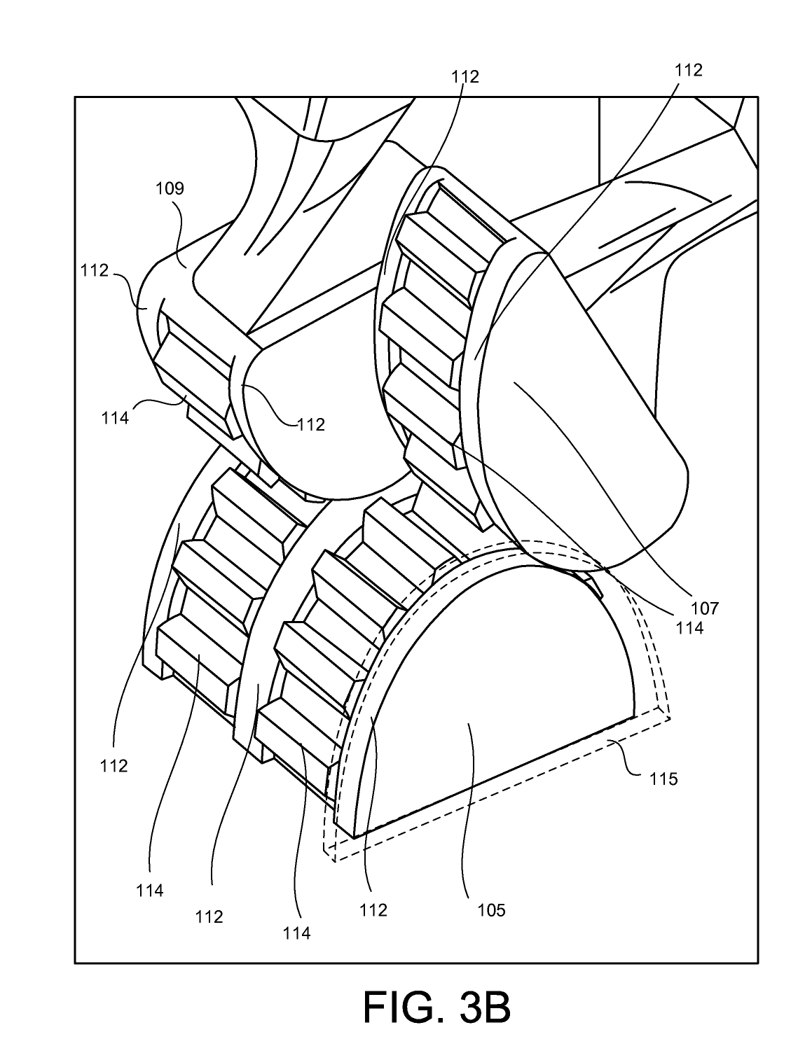

FIG. 1 illustrates a device 100 having a split rolling joint 104 coupled to a shaft 106, and coupled to first and second movable arms 108, 110 of a tool portion 102 according to an aspect. In some examples, the device 100 may be a surgical device used during a surgical procedure. In some examples, the device 100 may be used in Minimally Invasive Surgery (MIS) or laparoscopic surgical operations. FIG. 2 illustrates a second perspective of the split rolling joint 104 and the first and second movable arms 108, 110 according to an aspect. FIG. 3A illustrates a closer perspective of the split rolling joint 104 depicting the gear profile (which may alternately be considered a tooth profile or protrusion profile or arrangement of teeth, and like terms) on the curved surfaces of the split rolling joint 104 according to an aspect. FIG. 3B illustrates the perspective of the split rolling joint 104 of FIG. 3A but with an optional guide 115 (shown in dashed outline) to assist in guiding rotational movement of a movable arm according to an aspect.

Referring to FIGS. 1-3, the shaft 106 may be a circular cross section, elongated structure, such as a circular-cross-section tube. In other examples, the shaft 106 may have one or more non-circular-shaped cross section portions (e.g., oval, or various polygon shapes). In some examples, the shaft 106 may be a needle shaft or needle driver. In some examples, the shaft 106 may have a diameter between 1 mm and 5 mm. In some examples, the shaft 106 may have a diameter of 3 mm. In some examples, the shaft 106 may have a diameter of 4 mm. In some examples, the shaft 106 may include a handle disposed on or coupled to a proximal end portion of the shaft 106.

The tool portion 102 (e.g., a surgical end effector) may be any type of tool used for a surgical procedure. In some examples, the tool portion 102 may be a cutter or scissor. In some examples, the tool portion 102 may be a grasper configured to grasp another object (e.g., bodily tissue or another medical device). In still other examples, the tool portion 102 may perform other known surgical functions, such as fusing or stapling tissue, applying clips, cauterizing tissue, imaging tissue, and/or so forth. In some examples, the tool portion 102 may include one, two, or more than two movable portions. In some examples, the tool portion 102 may include a first movable arm 108 and a second movable arm 110. Moving the first moveable arm 108 and the second moveable arm 110 towards or away from each other allows the tool portion 102 to open and close, thereby performing a grasping or cutting function.

The split rolling joint 104 is coupled to an end portion of the shaft 106. Also, the split rolling joint 104 is coupled to the tool portion 102 having the first movable arm 108 and the second movable arm 110. The split rolling joint 104 includes a first curved portion 103 and a second curved portion 105. However, the first curved portion 103 is split into two independently controlled portions (e.g., a first split portion 107 and a second split portion 109). The first split portion 107 is disposed adjacent to the second split portion 109. The size and/or shape of the first and second split portions 107, 109 may be the same. In other examples, the size and/or shape of the first and second split portions 107, 109 are different. The first movable arm 108 is mounted on the second split portion 109. The second movable arm 110 is mounted on the first split portion 109.

The first and second curved portions 103, 105 may be any type of structure having a curved surface, such as right cylinders having various cross sectional shapes (e.g., circular, oval, elliptical, parabolic, etc., and divisions of such shapes). The shape and/or size of the first and second curved portions 103, 105 may be the same or different. In some examples, the first curved portion 103 and the second curved portion 105 are three-dimensional gear structures having curved surfaces.

In some examples, the first and second curved portions 103, 105 are cylindrical. For example, the first curved portion 103 and the second curved portion 105 may be one half of a cylinder (divided along its length) with one of the curved portions being further divided into the two independently controlled portions (upper segments), as shown in FIGS. 1-3, where the first and second movable arms 108, 110 are mounted. Because the first curved portion 103 is divided into two distinct portions, the general shapes of the first and second split portions 107, 109 may correspond to the shape of the first curved portion 103. As such, the first and second split portions 107, 109 may have any type of structure having a curved surface. In some examples, referring to FIG. 2, the second curved portion 105 (e.g., the segment that is not split) includes a curved surface portion 130 with edges 132, and a platform 134. The edges 132 may define the ends of the second curved portion 105. In some examples, the edges 132 may define a surface that is a semi-circle at each end of the second curved portion 105. However, the edges 132 may define a surface having other curved and non-curved shapes. The edges 132 may extend between (or be disposed between) the curved surface portion 130 and the platform 134. In some examples, the edges 132 are flat or substantially flat surfaces. In other examples, the edges 132 include one or more curved portions.

Referring to the second curved portion 105 of FIG. 2, the platform 134 may define a surface opposite to the curved surface portion 130 (e.g., the platform 134 may define a surface plane having a width and length). In some examples, the platform 134 may have a uniform width along its entire length. In other examples, the platform 134 may have multiple different widths. In some examples, the platform 134 may define a surface that is rectangular. In other examples, the platform 134 may define a surface having a non-rectangular shape. In other examples, the platform 134 includes projections or extensions that extend away from its surface (e.g., include one or more portions having a height or multiple heights). In some examples, the platform 134 may define a recess, hole, or cavity that extends into the second curved portion 105. In some examples, the platform 134 of the second curved portion 105 may be coupled to the tool portion 102.

Referring to FIG. 2, each of the first split portion 107 and the second split portion 109 includes a curved surface portion 140 with edges 136, and a platform 138. The platform 138 of the first split portion 107 may be coupled to the second movable arm 110, and the platform 138 of the second split portion 109 may be coupled to the first movable arm 108. The edges 136 of the first split portion 107 may define the ends of the first split portion 107, and the edges 136 of the second split portion 109 may define the ends of the second split portion 107. In some examples, the edges 136 of the first split portion 107 may define a surface that is a semi-circle at each end of the first split portion 107. Also, the edges 136 of the second split portion 109 may define a surface that is a semi-circle at each end of the second split portion 109. However, the edges 136 of each of the first split portion 107 and the second split portion 109 may define a surface having other curved and non-curved shapes. With respect to the first split portion 107, the edges 136 may extend between (or be disposed between) the curved surface portion 140 of the first split portion 107 and the platform 138 of the first split portion 107. With respect to the second split portion 109, the edges 136 may extend between (or be disposed between) the curved surface portion 140 of the second split portion 109 and the platform 138 of the second split portion 109.

Referring to FIGS. 1-3, the first and second split portions 107, 109 of the first curved portion 103 may be rollably (or movably) coupled to or engaged with the second curved portion 105 such that the first split portion 107 and the second split portion 109 independently roll (or otherwise move) with respect to the second curved portion 105. For example, the first movable arm 108 may move independently from the second movable arm 110 (and vice versa) such that movement of the first and second split portions 107, 109 with respect to the second curved portion 105 provides two degrees of movement.

In some examples, with respect to a mechanical relationship, the two degrees of movements include movement associated with the first split portion 107 rolling on the second curved portion 105 and movement associated with the second split portion 109 rolling on the second curved portion 105. With respect to a functional relationship, the two degrees of movement includes movement associated with the gripping function (e.g., opening or closing the first and second movable arms 108, 110) and rotational movement associated with the tool portion 102 (e.g., when the first and second arm members 110 move to positions to allow the grip to be pointed in various directions in a plane). For example, the tool portion 102 may be pointed in various directions (e.g., by moving the first and second movable arms 108, 110 as a unit), and the tool portion 102 may be opened and closed (e.g., by moving the first movable arm 108 with respect to the second movable arm 110.

As shown in FIGS. 2 and 3A, the curved surface portion 130 of the second curved portion 105 includes two adjacent rows of gear profiles 114. The curved surface portion 140 of the first split portion 107 includes one row of the gear profile 114, and the curved surface portion 140 of the second split portion 109 includes one row of the gear profile 114. In some examples, the gear profile 114 may include a plurality of recesses and protrusions that are disposed within a row over the curved surface. However, the gear profile 114 may have any type of gear profile structure (e.g., various pitches, tooth heights, tooth widths, straight cut, helical cut, etc.). In some examples, the gear profile 114 of the first split portion 107 is the same as the gear profile 114 of the second split portion 109. In other examples, the gear profile 114 of the first split portion 107 is different than the gear profile 114 of the second split portion 109. Also, the size or shape of the gear profiles 114 of the first and second split portions 107, 109 may be the same or different. The row of the gear profile 114 of the first split portion 107 is configured to engage with one of the two rows of gear profiles 114 of the second curved portion 105 such that the first split portion 107 can rotate around the second curved portion 105, and the single row of gear profile 114 of the second split portion 109 is configured to engage with the other of the two rows of the second curved portion 105 such that the second split portion 109 can rotate around the second curved portion 105. Also, the design of the gear profiles may prevent or minimize slip between the two engaging segments and may allow precise control of the gripper locations.

Also, the curved surface portion 140 of the first split portion 107, the curved surface portion 140 of the second split portion 109, and the curved surface portion 130 of the second curved portion 105 may include non-geared areas 112 which support the compressive loads associated with the motion of the tool portion 102 and holding the assembly together. For example, referring to the second curved portion 105 of FIG. 3A, there are three distinct non-geared areas 112. These non-geared areas 112 are included in the design so that all (or most of) of the compressive loads are transferred away from the gear profile 114 which would otherwise experience higher stresses because of smaller cross-sections and stress concentrations. In some examples, the gears do not contact the bottoms of the recesses associated with the gears on the other component. For instance, the top land of one geared portion does not contact the bottom land of the opposite geared portion. The non-geared areas 112 may be designed to maintain proper spacing between the geared portions. In other words, the pitch circles of the two geared portions are tangent to one another. The pitch circles of the geared portions can have the same diameter or different diameters. In either case, the non-geared portions 112 may be designed such that the distance between the centers of the gears is half the pitch diameter of one gear plus half the pitch diameter of the other gear. Also, referring to FIG. 3B, one or both of the side portions of the second curved portion 105 may include a guide 115 to assist in reducing lateral slip of the split portions off the sides of the bottom portion and/or to assist in guiding rotational movement of the joint. The guide 115 is depicted as a dashed line to not obscure the other components of FIG. 3B. The guide 115 may be a channel, recess, protrusion, or portion that extends from (into) the second curved portion 105 in order to guide rotational movement of at least one of the first and second split portions 107, 109 with respect to the second curved portion 105. In some examples, at least one of the first and second split portions 107, 109 includes corresponding guide features configured to engage with the guide 115.

In some examples, the split rolling joint 104 may be a modification of a Compliant Rolling-Contact Element (CORE) joint. For instance, the split rolling joint 104 may have truncated joints to reduce the size of the joint. Consequently, this also limits the range of motion to approximately ±90 degrees which is considered acceptable for many applications. However, the split rolling joint 104 may have a range of motion different than ±90 degrees. In some examples, the range of range of motion in one direction may be different than the range of motion in the opposite direction. Also, in place of flexures, the split rolling joint 104 may use the input actuation force to maintain compressive contact between the first curved portion 103 and the second curved portion 105, as further discussed below.

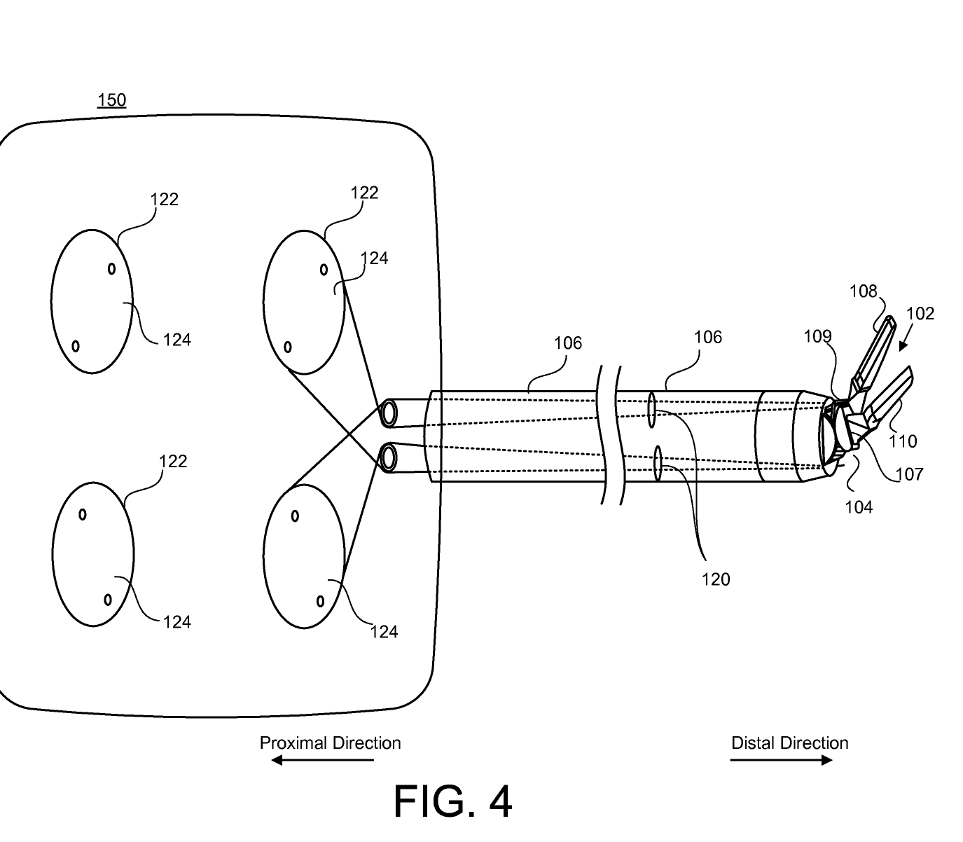

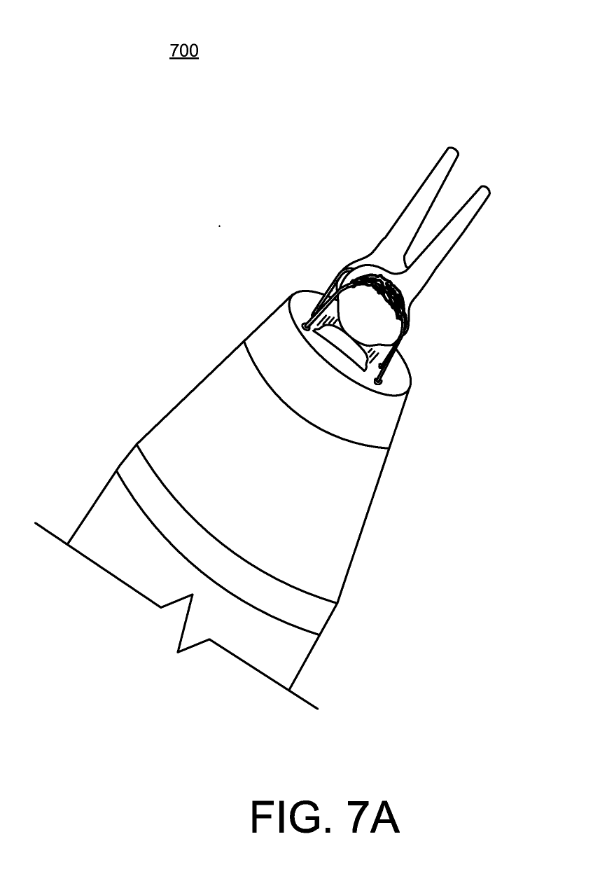

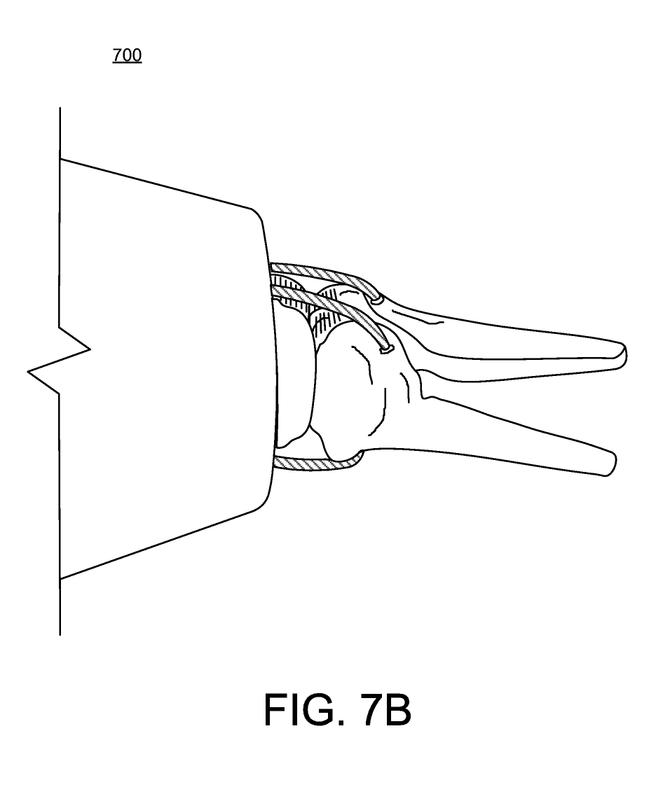

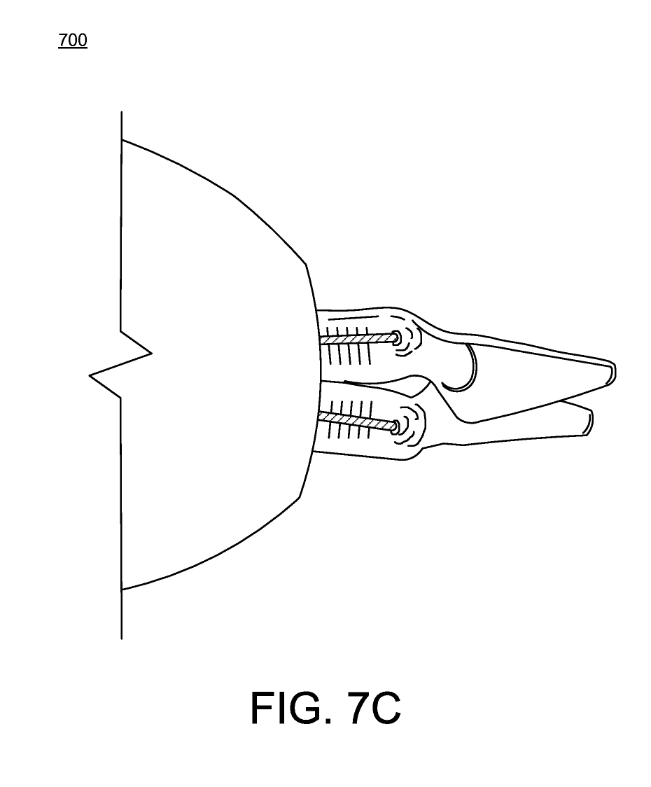

FIG. 4 illustrates a device 150 depicting an actuation mechanism for controlling the two-degree-of-freedom tool portion 102 according to an aspect. For example, an actuation member 120 may be coupled to the first movable arm 108 (or the second split portion 109) and to an actuator (e.g., actuation spools 122), and another actuation member 120 may be coupled to the second movable arm 110 (or the first split portion 107) and coupled to the actuation spools 122. In some examples, the actuation members 120 may include actuation cables or wires. The actuation spools 122 may be operated via a robotic control interface 124 which places input force on the first and second movable arms 108, 110 (or the first and second split portions 107, 109) in order to open and close the tool portion 102 as well as rotate the tool portion 102 with respect to the shaft 106. Further, the actuation members 120 maintain compressive contact between the first curved portion 103 and the second curved portion 105 such that flexures are not needed. In some examples, two opposing actuation members 120 are attached to one actuation spool 122 such that when the actuation spool 122 rotates in one direction, the actuation spool 122 applies tension to one actuation member 120 and slackens or pushes the opposing actuation member 120. Each actuation spool 122 may control one of the degrees of freedom, so only two actuation spools 112 depicted in FIG. 4 are used for the two degrees of freedom of the split core wrist mechanism. However, additional actuation spool 120 may be incorporated into the device (e.g., the two activation spools 122 on the left side of the figures) for other degrees of freedom. In some examples, the device 150 may include any number of activation spools 122 and any number of degrees of freedom within the instrument. As an example, referring to FIG. 7B, the actuation cable in the foreground (controlling the bottom arm) is a single cable with both ends attached to a single spool. Therefore, rotating the spool would tighten one side of the cable and move it to the proximal end of the instrument, while slackening and allowing the other end of the cable to move to the distal end of the instrument.

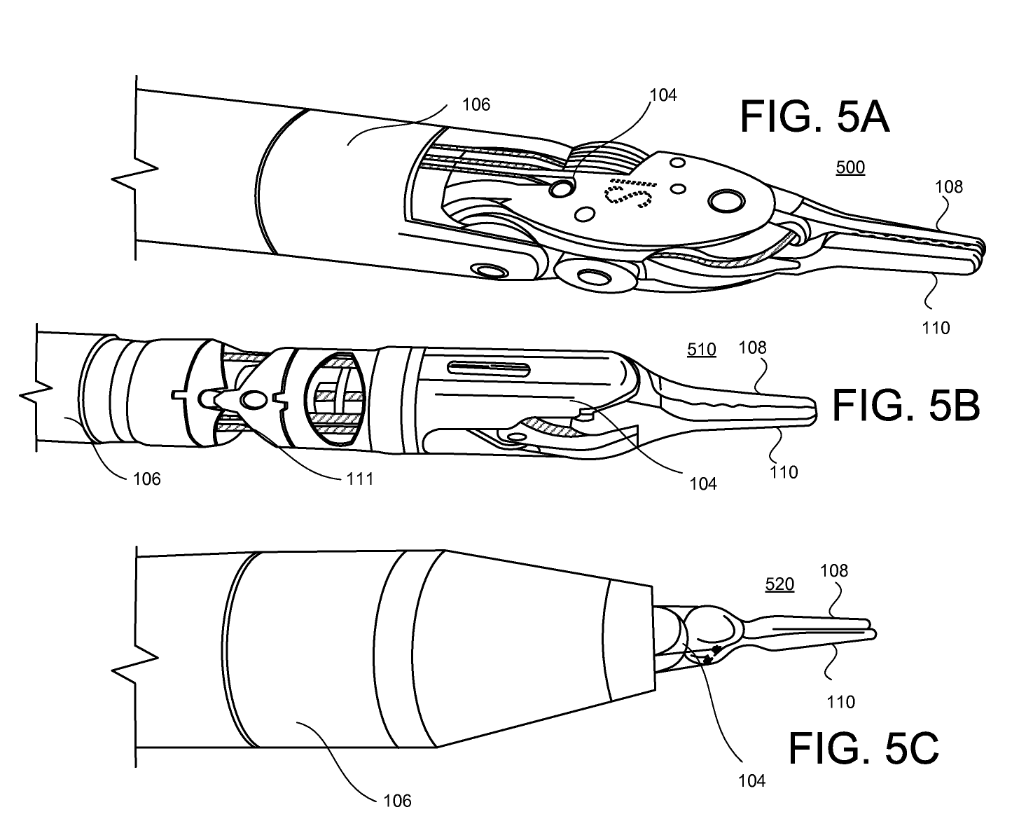

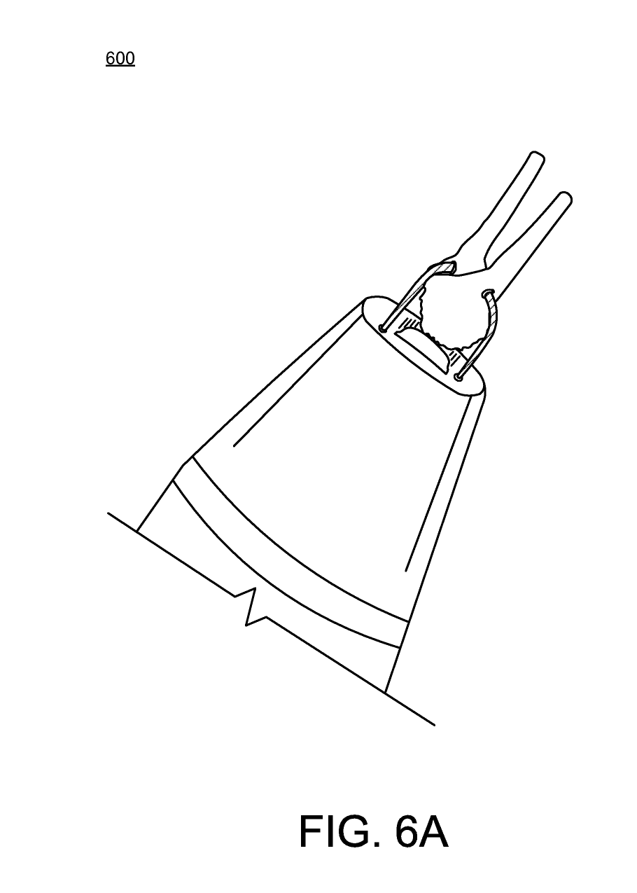

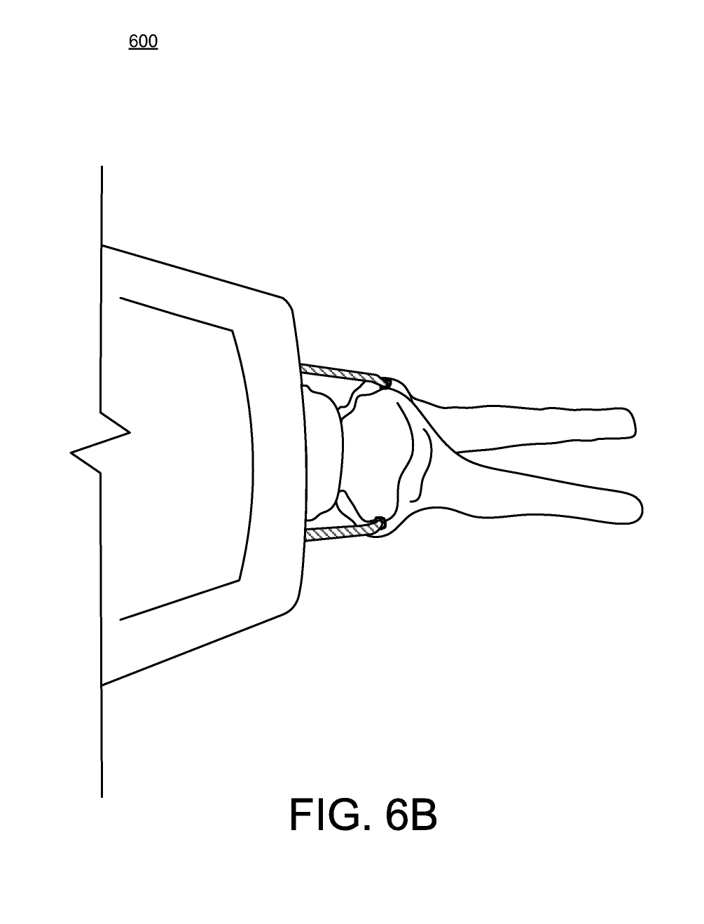

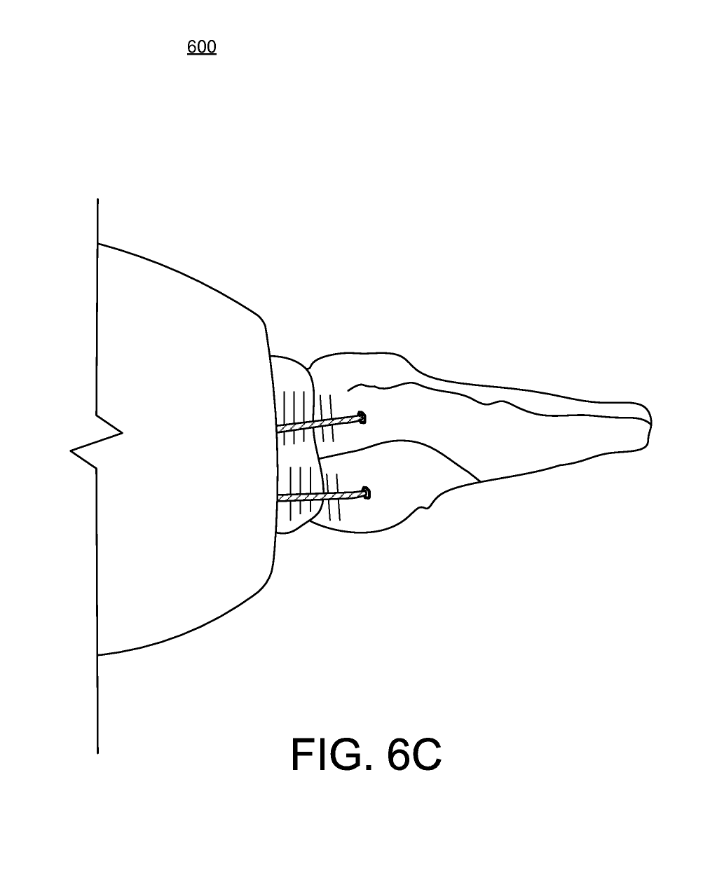

FIGS. 5-7 illustrate various aspects of the devices 100, 150 discussed with reference to FIGS. 1-4. FIG. 5A illustrates a device 500 having the first movable arm 108, the second movable arm, the split rolling joint 104, and the shaft 106 according to an aspect. FIG. 5B illustrates a device 510 having the first movable arm 108, the second movable arm, the split rolling joint 104, the shaft 106, and an articulation joint 111 according to an aspect. FIG. 5C illustrates a device 520 having the first movable arm 108, the second movable arm, the split rolling joint 104, and the shaft 106 according to an aspect. FIG. 6A illustrates a device 600 according to an aspect. FIG. 6B illustrates the device 600 according to another aspect. FIG. 6C illustrates the device 600 according to another aspect. FIG. 7A illustrates a device 700 according to an aspect. FIG. 7B illustrates the device 700 according to another aspect. FIG. 7C illustrates the device 700 according to another aspect.

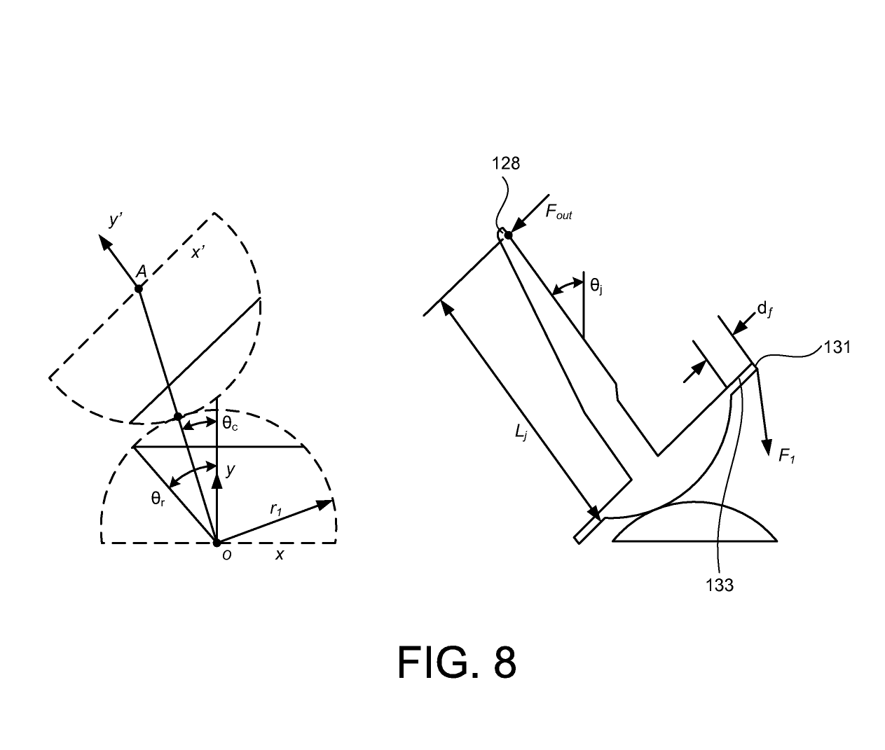

FIG. 8 illustrates the geometry and parameters used in deriving equations of motion and force output for the split rolling joint 104 of FIGS. 1-7 according to an aspect. Referring to FIG. 8, a design analysis may define several positions of the jaws (e.g., the first and second movable arms 108, 110 of FIGS. 1-7) and the relationship between the input and output forces of the split rolling joint 104 and the tool portion 102 of FIGS. 1-7 (e.g., also referred to as Split CORE mechanism). The output force is defined as the force acting orthogonally (or substantially orthogonal) to a tip portion 128 of the jaw (e.g., either the first or second movable arms 108, 110 of FIGS. 1-7) while the input force is defined as a force at an edge portion 131 of the top half of the joint and acts toward the edge of the lower segment. The tip portion 128 may be a distal end portion of the jaw (e.g., the distal end portion of the first movable arm 108 or the distal end portion of the second movable arm 108 of FIG. 1). In some examples, the top half of the joint (e.g., the first split portion 107 or the second split portion 109 of FIGS. 1-7) may define a protrusion 133 that extends away from the cylinder portion of the joint. The protrusion 133 may have a length of d_{f}, where the tip portion 128 is disposed at the end of the protrusion 133.

The output force models the reaction force applied by an object of the mechanism, which may be gripping or grasping. The input force is provided by the actuation members 120 from the mechanism through the shaft 106 and to the base housing where the input torque is provided.

The motion of the Split CORE mechanism can be modeled when compared to the motion of the traditional CORE joint. The traditional CORE joint is modeled as two half cylinders—a fixed lower segment and a free upper segment which rolls along the curved surface of the lower segment. This is shown in FIG. 8 by the dashed lines. The design of the Split CORE mechanism is based on the same principle, using a half cylinder surface, or some smaller portion of the circular arc to reduce the size of the joint as shown in FIG. 8 by the solid lines. In some examples, the arcs of both the traditional CORE model and the Split CORE mechanism have the same (or similar) radius of curvature, r₁, and are (or can be) concentric. The centers of the lower segment (e.g., the second curved portion 105) and the upper segment (e.g., one of the first and second split portions 107, 109) are labeled as O and A, respectively. If using some smaller portion of the circular arc, neither of the centers physically exists on the Split CORE mechanism, but are still used as reference points because they simplify the derivations of equations of motion and force output. The angle θᵣ is used to describe the size of the arc used in the design. For example, if θᵣ is equal to 90°, the result may be equivalent to the traditional CORE joint. If θᵣ is equal to 45° the resulting mechanism may look similar to the one shown by solid lines in FIG. 8.

The parameters of interest in this design are the output force at the jaws, Fₒᵤₜ, the angle of the jaws, θⱼ, and the required input forces, F₁ and F₂. The principle of virtual work is used to determine these input forces for any given values of Fₒᵤₜ and θⱼ. The angle used to describe the point of contact between the upper and lower segments (θ_{c}) is also used but can be described as a function of the jaw angle by the following relation:

$${θ}_{c} = \frac{{θ}_{j}}{2} \qquad Eq . ( 1 )$$

All angles shown in FIG. 8 are defined as positive counter-clockwise from the y-axis, and the origin of the coordinate system is at point O as shown. Another coordinate system, x′-y′, is also shown. This system will be used along with a rotation matrix to define the location and direction of the input forces in terms of the x-y coordinate system. The origin of the x′-y′ coordinate system is point A.

Two input forces exist in this design: F₁ and F₂. An assumption can be made regarding the relationship between these two forces. If the actuation members 120 attached at the points of F₁ and F₂ are connected to a common actuation spool 122, then as a force is applied to one actuation member 120, the force in the opposite actuation member 120 goes to zero. For example, under this assumption, if F₁ equals 2N, then F₂ is zero. In addition to this assumption, FIG. 8 shows that for any nonzero value of Fₒᵤₜ, F₁ will also be nonzero, and consequently F₂ will be zero. This is because F₁ is the only force that can balance the system. If considering the other jaw in the assembly (not shown in FIG. 8), for any nonzero value of Fₒᵤₜ, F₂ would be nonzero and F₁ would be zero. The derivations that follow apply to the case shown in FIG. 8 where F₂ is zero. However, the same approach can be used to consider the case for the opposite jaw.

The method of virtual work can be used to determine the magnitude of F₁ for given values of Fₒᵤₜ and θⱼ. The first step in calculating the virtual work in the system is choosing a generalized coordinate. The jaw angle, θⱼ, is a convenient parameter because it is used to describe the position of the jaw, and because the expression for F₁ will be derived as a function of θⱼ. Therefore, θⱼ will be used as the generalized coordinate. Next, each of the applied forces is written in vector form in terms of the generalized coordinate. The input force in this model is placed at a distance d_{f} from the corner of the upper segment and points toward a point a distance d_{f} from the corresponding corner of the lower segment. This assumption is based on the idea that actuation members 120 provide the input forces and route around the lower geometry before entering the shaft 106 and connecting to the robotic control interface 124 at the opposite end of the shaft 106. The reason for placing the input force a distance from the corner is to increase the moment arm, and consequently the mechanical advantage. This may be important when the point of rolling contact is near corners of the segments (i.e. as θ_{c} approaches θᵣ). However, in this configuration, it is also important to address any interference that may result from placing the forces and the actuation members 120 at these locations. Using this assumption, the directions of the forces are shown in Eqs. (2) and (3) for Fₒᵤₜ and F₁, respectively.

$$\begin{matrix}{F}_{out} = {F}_{out} ( - cos {θ}_{j} \hat{i} - sin {θ}_{j} \hat{j} ) & Eq . ( 2 ) \\ {F}_{1} = {F}_{1} ( sin \frac{{θ}_{j}}{2} \hat{i} - cos \frac{{θ}_{j}}{2} \hat{j} ) & Eq . ( 3 )\end{matrix}$$

Next, position vectors are written from the origin, O, to each of the applied forces. The vector describing Fₒᵤₜ is fairly simple to describe in terms of θⱼ and is given by Eq. (4). The other vector is more complicated because it lies at some point on the arc determined by θᵣ and that point sits somewhere in space determined by θⱼ. To simplify the derivation of the position vector F₁, two vectors can be summed together—one from point O to point A, and the second from point A to the location of force application. This second vector can be described in the x-y coordinate system using a rotation matrix. This results in the following equation for the position of F₁.

$$\begin{matrix}{Z}_{out} = [ - 2 {r}_{1} sin \frac{{θ}_{j}}{2} - ( {L}_{j} - {r}_{1} cos {θ}_{r} ) sin {θ}_{j} ] \hat{I} + [ 2 {r}_{1} cos \frac{{θ}_{j}}{2} + ( {L}_{j} - {r}_{1} cos {θ}_{r} ) cos {θ}_{j} ] \hat{I} & Eq . ( 4 ) \\ {Z}_{1} = - 2 {r}_{1} sin \frac{{θ}_{j}}{2} \hat{i} + 2 {r}_{1} cos \frac{{θ}_{j}}{2} \hat{j} + [ \begin{matrix}cos {θ}_{j} & - sin {θ}_{j} \\ sin {θ}_{j} & cos {θ}_{j}\end{matrix} ] [ \begin{matrix}{r}_{1} sin {θ}_{r} \hat{i} \\ - {r}_{1} cos {θ}_{r} \hat{j}\end{matrix} ] & Eq . ( 5 )\end{matrix}$$

Eq. (5) can be expanded to its î and ĵ components and then simplified—which results in Eq. (6).

$${z}_{1} = [ - 2 {r}_{1} sin \frac{{θ}_{j}}{2} + {r}_{1} sin ( {θ}_{j} + {θ}_{r} ) ] \hat{i} + [ 2 {r}_{1} cos \frac{{θ}_{j}}{2} = {r}_{1} cos ( {θ}_{j} + {θ}_{r} ) ] \hat{j} \qquad Eq . ( 6 )$$

The next step is to determine the virtual displacement of each point of force application by calculating the partial derivatives of Eqs. (4) and (6) with respect to the generalized coordinate.

$$\begin{matrix}δ {Z}_{out} = \{ [ - {r}_{1} cos \frac{{θ}_{j}}{2} - ( {L}_{j} - {r}_{1} cos {θ}_{r} ) cos {θ}_{j} ] \hat{i} + [ - {r}_{1} sin \frac{{θ}_{j}}{2} - ( {L}_{j} - {r}_{1} cos {θ}_{r} ) sin {θ}_{j} ] \hat{j} \} {δθ}_{j} & Eq . ( 7 ) \\ δ {Z}_{1} = \{ [ - {r}_{1} cos \frac{{θ}_{j}}{2} + {r}_{1} cos ( {θ}_{j} + {θ}_{r} ) ] \hat{i} + [ - {r}_{1} sin \frac{{θ}_{j}}{2} + {r}_{1} sin ( {θ}_{j} + {θ}_{r} ) ] \hat{j} \} {δθ}_{j} & Eq . ( 8 )\end{matrix}$$

The virtual work associated with each force is determined by calculating the dot product of each force vector (Eqs. (2) and (3)) and its respective virtual displacement vector (Eqs. (7) and (8)).

$$\begin{matrix}δ {W}_{out} = {F}_{out} ( {r}_{1} cos \frac{{θ}_{j}}{2} - {r}_{1} cos {θ}_{r} + {L}_{j} ) {δθ}_{j} & Eq . ( 9 ) \\ δ {W}_{1} = - {F}_{1} {r}_{1} sin ( {θ}_{r} + \frac{{θ}_{j}}{2} ) {δθ}_{j} & Eq . ( 10 )\end{matrix}$$

The total virtual work in the system is calculated by summing each component of virtual work from Eqs. (9) and (10). For a system in equilibrium, the principle of virtual work states that the total virtual work is equal to zero. This makes it possible to rearrange the equation to determine F₁ for various values of Fₒᵤₜ and θⱼ.

$$\begin{matrix}0 = [ {F}_{out} ( {r}_{1} cos \frac{{θ}_{j}}{2} - {r}_{1} cos {θ}_{r} + {L}_{j} ) - {F}_{1} {r}_{1} sin ( {θ}_{r} + \frac{{θ}_{j}}{2} ) ] & Eq . ( 11 ) \\ {F}_{1} = \frac{{F}_{out} ( cos \frac{{θ}_{j}}{2} - cos {θ}_{r} + \frac{{L}_{j}}{{r}_{1}} )}{sin ( {θ}_{r} + \frac{{θ}_{j}}{2} )} & Eq . ( 12 )\end{matrix}$$

It may be desirable in some embodiments to apply a certain amount of preload force to the points of force application (FIG. 8). This reduces effects (e.g., any effects) of backlash that may occur and ensures higher levels of control over the motion of the mechanism. If an equal preload force is applied to both sides of the mechanism (i.e., equal preload in both actuation members 120), then the changes to the previous derivations are relatively simple. The input force term F₁ in Eq. (11) is replaced by (F₁+Fₚ), where Fₚ is the preload force. The virtual work derivation would also include the effects of Fₚ at the location of F₂. Doing this results in a slightly different expression for F₁ given by:

$${F}_{1} = \frac{{F}_{out} ( cos \frac{{θ}_{j}}{2} - cos {θ}_{r} + \frac{{L}_{i}}{{r}_{1}} ) - 2 {F}_{p} {r}_{1} cos {θ}_{r} sin \frac{{θ}_{j}}{2}}{sin ( {θ}_{r} + \frac{{θ}_{j}}{2} )} \qquad Eq . ( 13 )$$

This new expression for F₁, which includes a preload force on the system, shows two interesting behaviors that occur. First, by including a preload force on both actuation members 120, the required input force is reduced when θⱼ is between 0° and 90°, but is increased when θⱼ is between 0° and −90°. Second, for θᵣ=90° the preload force has no effect on the required input force and Eq. (13) becomes equivalent to Eq. (12).

To demonstrate the use of these equations of motion, consider a design where the desired jaw rotation is ±90° with a jaw length of 6.25 mm and a desired output force of 2 N. Assume that there is not a preload force in the actuation members 120. To achieve this motion θᵣ must be at least 45°. To provide reasonable structural support at the extremes of motion, θᵣ=60°. In this example, the instrument may be designed to fit within a 3 mm circle so that it can be attached to a 3 mm shaft. To do this, the base of Split CORE joint may be assumed to be square. Therefore, one side of the square is equal to 2r₁ sin θᵣ. The diagonal of the square will be equal to the diameter of the desired shaft size (3 mm). Using this information r₁ is calculated as follows:

$$\begin{matrix}{( 3 mm )}^{2} = 2 {( 2 {r}_{1} sin {θ}_{r} )}^{2} & Eq . ( 14 ) \\ {r}_{1} = \sqrt{\frac{g}{8 {sin}^{2} {θ}_{r}}} & Eq . ( 15 ) \\ {r}_{1} = 1.23 mm & Eq . ( 16 )\end{matrix}$$

The distance from the upper segment to the point of force application (d_{f}) may also be determined in this design. One option is to define this distance as the point where the force would be applied if θᵣ were equal to 90°. Doing this gives the design the same mechanical advantage as a traditional CORE mechanism, but its overall height is reduced because the actual profile is defined by θᵣ=60°. Therefore, calculating d_{f} is done using the following relation:

d_{f}=r₁−r₁ sin θᵣ Eq. (17):

d_{f}=0.165 mm Eq. (18):

With these values the input force, F₁, can be determined for any jaw rotation using Eq. (12). In this calculation the value of θᵣ=90° will be used because that defines the location of force input. For other calculations such as segment height and range of motion, θᵣ=60° would be used.

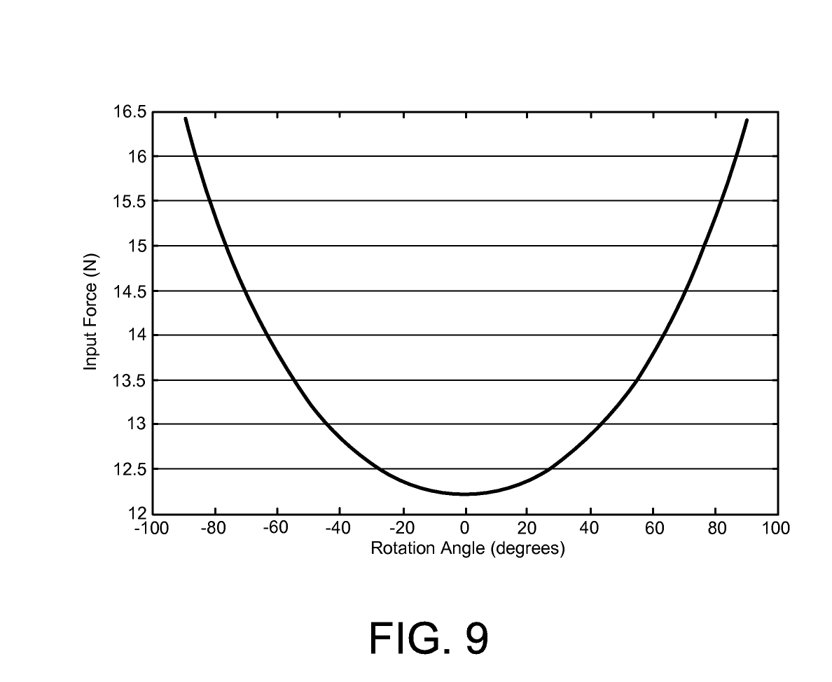

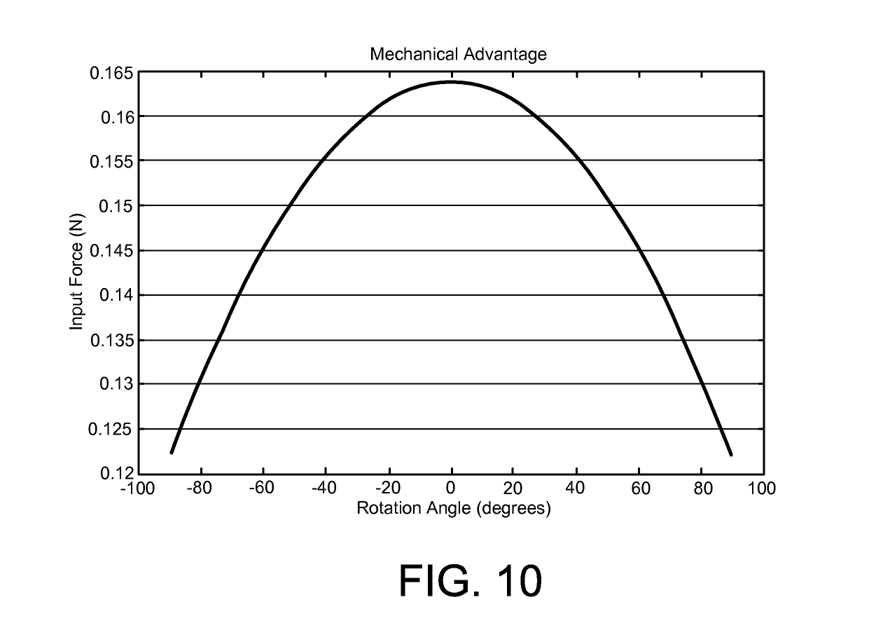

FIG. 9 illustrates a graph depicting the required input force for a range of θⱼ from −90° to 90° for the split rolling joint according to an aspect. This plot shows that the required force is symmetric about θⱼ=0° and ranges between approximately 12 and 16 N. The locations of greatest force are at the extremes of motion. This is to be expected because it is where the moment arm of force application is minimized. FIG. 10 illustrates a graph of the mechanical advantage of the split rolling joint 104. Also, FIG. 10 illustrates this concept where mechanical advantage is maximum at θⱼ=0°.

In addition to the force requirements, mechanical advantage also gives some insight into the control and precision of the instrument. Mechanical advantage can be used to describe the relationship between input displacement and output displacement. In this particular design, the input displacement is the amount of motion in the actuation cable. The output displacement corresponds to the displacement of the tip of the jaw where Fₒᵤₜ is positioned (see FIG. 8). For the example design given, this means that when θⱼ=0° (where M.A.=0.164) a 1 mm displacement of the actuation member 120 would result in an output displacement of approximately 6.10 mm. This is based on the following relationship:

$$MA = \frac{input displacement}{output displacement} \qquad Eq . ( 19 )$$

There are a few different ways to maximize precision and control of the instrument tip. One way is to increase the mechanical advantage of the system. This can be done by increasing the radius of curvature in the upper and lower segments (r₁). Another way to accomplish improved control is to reduce the diameter of the actuation spool 122 which is used to actuate the actuation member 120. With a smaller diameter actuation spool 122, a given rotational input will result in a smaller cable displacement than would occur with the same rotational input on a larger spool. This method does not change the required input force (or mechanical advantage) but it does improve the control of the motion at the jaw tip.

The critical stresses experienced by the Split CORE mechanism can be determined using Hertzian Contact Stress Theory. Contact stress theory is used to model the interfacial stresses between two mating solids. In the case of two cylindrical surfaces, the area of contact forms a rectangle of width 2b and length l. The length, l, is simply the total length of the flat regions carrying the compressive loads. Using the parameters shown in FIG. 8, the half width of the stress area, b, is given by the following equation:

$$b = \sqrt{\frac{4 {r}_{1} F ( 1 - {v}^{2} )}{π lE}} \qquad Eq . ( 20 )$$

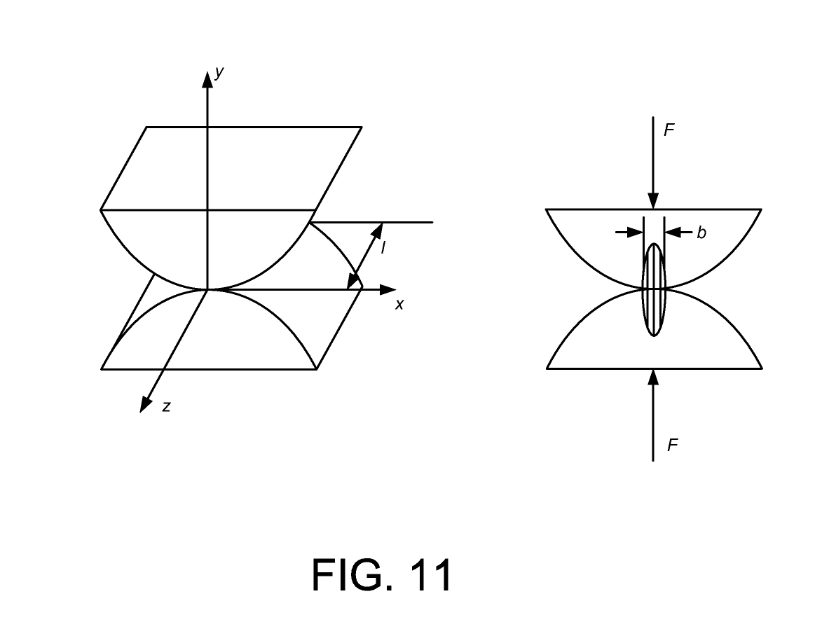

The parameter F is the input force F₁ or F₂, depending on which case is being considered, ν is Poisson's ratio, and E is the modulus of elasticity for the material being used. Eq. (20) assumes that the radius of curvature for upper and lower segments is equal and that both are of the same material. The contact area creates an elliptical pressure distribution with its maximum at the center. FIG. 11 illustrates the geometry depicting the parameters used to determine the stress states cause by the contact between the upper and lower segments of the Split CORE design. Also, the distribution is shown in the right side of FIG. 11. The maximum pressure is defined as:

$${P}_{max} = \frac{2 F}{π bl} \qquad Eq . ( 21 )$$

Subsequently, the stress states along each of the three axes can be expressed in terms of the distance away from the point of contact, or the depth into the material. This depth is denoted as y, as it corresponds to the y axis. These expressions are given by the following three equations.

$$\begin{matrix}{σ}_{x} = - {P}_{max} ( \frac{1 + 2 {( \frac{y}{b} )}^{2}}{\sqrt{1 + {( \frac{y}{b} )}^{2}}} - \vert \frac{y}{b} \vert ) & Eq . ( 22 ) \\ {σ}_{y} = \frac{- {P}_{max}}{\sqrt{1 + {( \frac{y}{b} )}^{2}}} & Eq . ( 23 ) \\ {σ}_{z} = - 2 v {P}_{max} ( \sqrt{1 + {( \frac{y}{b} )}^{2}} + \vert \frac{y}{b} \vert ) & Eq . ( 24 )\end{matrix}$$

The parameters used in the previous example will be used here to determine the stress states at the contact point of the mechanism. For this design, the material being used is titanium (Ti-6Al-4V) with an elastic modulus of 114 GPa, compressive yield strength of 1070 MPa, and Poisson's ratio of 0.34. The non-geared portion of the contact surface may be one third of the total length of the joint, where the length of the joint is equal to 2r₁, or 2.12 mm, so that it fits on a 3 mm instrument shaft. The remaining portion of the surfaces is comprised of gear teeth which only transmit loads associated with motion. From this information the length, l, is calculated to be approximately 0.7 mm and the contact width, b is calculated using Eq. (20). These calculations are based on the position at which θⱼ is zero, which corresponds to F₁=12.2 N. However, this same approach can be used to determine the contact stresses at any angle of rotation.

$$\begin{matrix}b = \sqrt{\frac{4 ( 0.00123 ) ( 12.2 ) ( 1 - {0.34}^{2} )}{π ( 0.0007 ) ( 144 \times {10}^{9} )}} & Eq . ( 25 ) \\ b = 0.015 mm & Eq . ( 26 )\end{matrix}$$

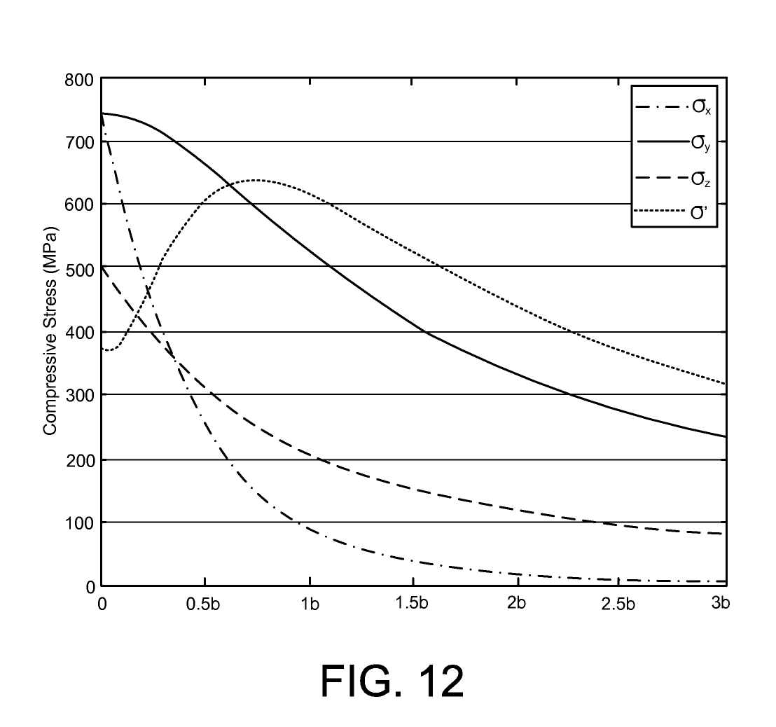

These values are substituted into Eq. (21) to calculate the contact pressure which gives a value of Pₘₐₓ=742 MPa. Lastly, these values are substituted into Eqs. (22)-(24) to determine each of the stress states. FIG. 12 illustrates a graph plotting the stress states at the contact point when the example design is in the vertical position according to an aspect. The maximum stress in each of the three directions occurs at the outer surface where contact is made. The maximum stresses for σₓ, σ_{y}, and σ_{z} at this location are 742 MPa, 742 MPa, and 504 Mpa, respectively. This results in a maximum Von Mises stress of σ′=638 MPa. The location of the maximum Von Mises stress is approximately 0.011 mm from the contact surface (z≈0.74b). This gives a minimum safety factor of 1.68.

The motion of the Split CORE gripper mechanism, as described in some implementations, is straightforward and predictable. Given a desired rotation angle and output force, the required input forces can be calculated. Also, because the jaw segments roll, rather than slide, along the surface of the lower segment the effects of friction are minimal. The critical stresses in the system are due to compressive contact and occur at rolling contact between upper and lower segments. These stresses can also be predicted for a particular rotation angle and output force.

FIG. 13 illustrates a device 200 according to another aspect. The device 200 includes a rolling arm 208 configured to move with respect to a fixed arm 210. The rolling arm 208 may have a curved portion 216 that is configured to roll or rotate on the curved portion 205 in a manner previous explained. The curved portion 216 may be the first curved portion 105, the first split portion 107, or the second split portion 109 of FIGS. 1-7. The fixed arm 210 may be fixedly coupled to the curved portion 205 (or the shaft, or another component of the device 200). For instance, the fixed arm 210 may not move with respect to the curved portion 205. The curved portion 205 may be the same or similar to the second curved portion 105 explained with reference to the previous figures. The curved portion 205 may be coupled to a shaft such as the shaft 106 of the previous figures.

Also, the rolling arm 208 may include an arm extension 218 that extends from the curved portion 216 of the rolling arm 208. In some examples, the arm extension 218 may extend from the curved portion 216 at an angle θₐ. For example, the curved portion 216 may define a surface 220 disposed opposite to the curved surface of the curved portion 216. In some examples, the surface 220 may be non-curved or linear (e.g., devoid of curvature). In some examples, the surface 220 may be the top of the curved portion 216. Also, the arm extension 218 may define a surface 214 that is opposite to a surface 212 of the fixed arm 210. In some examples, the surface 214 of the arm extension 218 faces the surface 212 of the fixed arm 210. When the rolling arm 208 rolls on the curved portion 205, the surface 214 of the arm extension 218 moves closer or further away from the surface 212 of the fixed arm 210. The surface 214 of the arm extension 218 and the surface 220 of the curved portion 216 may form the angle θₐ. In some examples, the angle θₐ may be an obtuse angle. In other examples, the angle θₐ may be an acute angle. In other examples, the angle θₐ may be substantially 90 degrees.

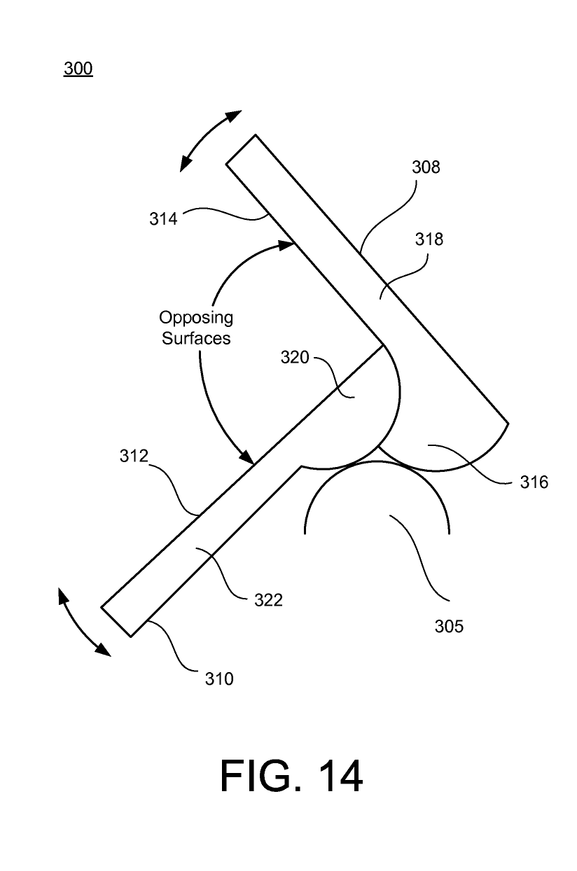

FIG. 14 illustrates a device 300 according to another aspect. The device 300 may include first and second rolling arms 308, 310 rollably coupled to a curved portion 305 as described with reference to the previous figures. For example, the rolling arms 308, 310 are similar to the description above, but the arm angle is offset from the rolling arms 308, 310. The device 300 may have a unique advantage in that for a given jaw-closed angle from the shaft axis, the distance to the tip (e.g., tips of 308, 310) is less than it would be for the straight arm configuration. In some examples, the device 300 may have its jaws offset 90 degrees from the axis of the instrument. This “throw distance” (e.g., the distance from the shaft's center axis to the tip of the tool) may be important in very tight surgical spaces. In some examples, this configuration may allow the device 300 to work on the inside walls of a confined cylinder because the tips of the jaws are relatively close to the main axis of the instrument. Also, in some examples, this offset angle can be implemented in the single fixed arm configuration as shown in FIG. 13.

The first rolling arm 308 may include a curved portion 316 configured to roll on a surface of the curved portion 305, and a first arm extension 318 that extends from the curved portion 316. The second rolling arm 310 may include a curved portion 320 configured to roll on the surface of the curved portion 305, and a second arm extension 322 that extends from the curved portion 320. In some examples, the curved portion 316 may have a cylindrical shape that is the same as the curved portion 320. In other examples, the curved portion 316 has a cylindrical shape that is different from the curved portion 320. The first arm extension 318 includes a surface 314 that is opposite to a surface 312 of the second arm extension 322.

Also, in some examples, any of the previously described devices may include a wrist mechanism between the shaft and the joint. In some examples, the wrist mechanism may include a one- or two-DOF wrist mechanism between the shaft and the joint. The wrist mechanism could be of various types commonly known in the art.

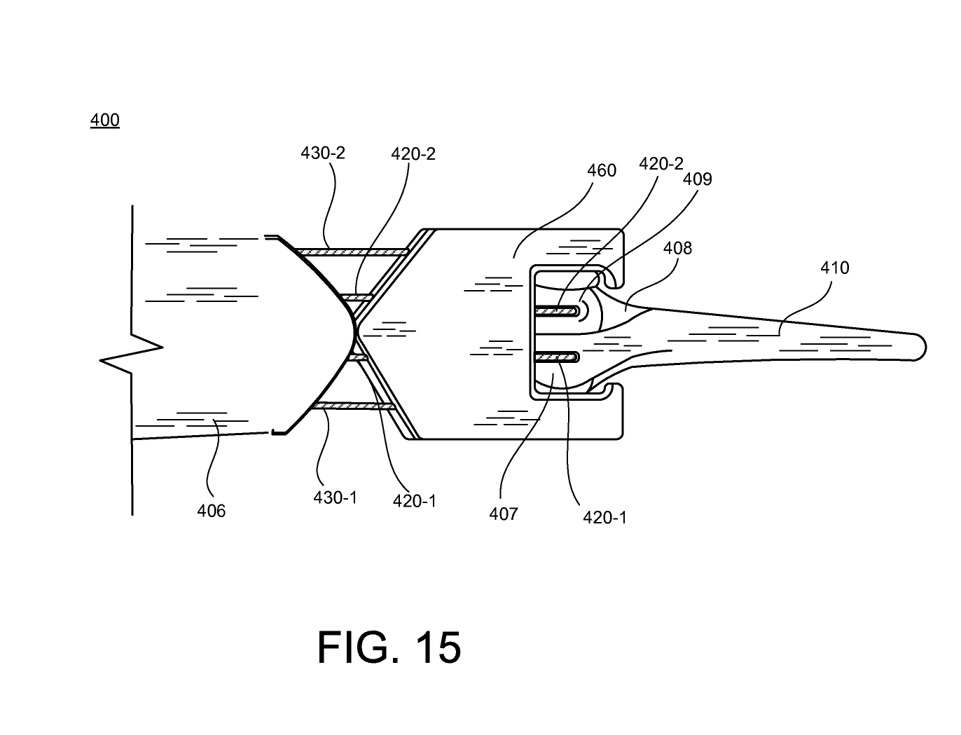

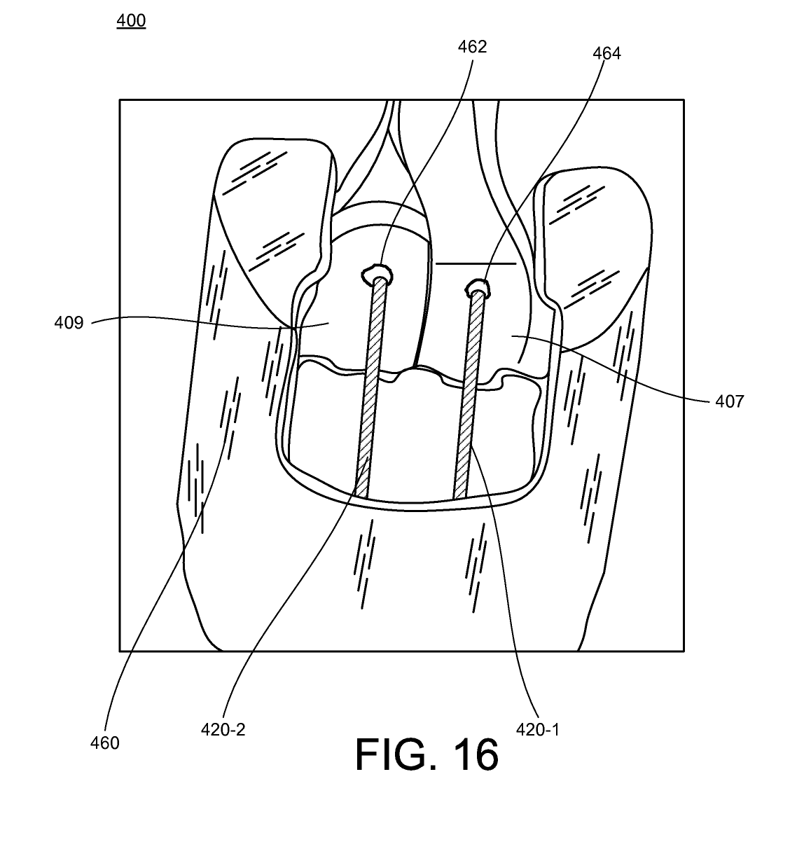

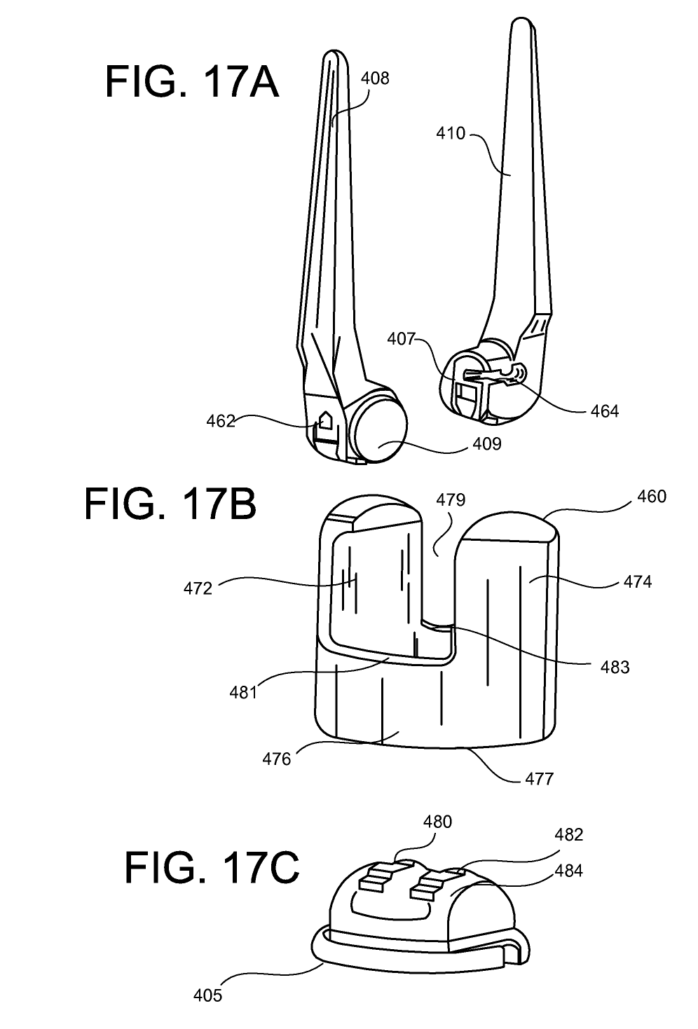

FIGS. 15-17 illustrate a device 400 according to an aspect. FIG. 15 illustrates a side view of the device 400 according to an aspect. FIG. 16 illustrates a perspective of the device 400 according to an aspect. FIGS. 17A-B illustrate separated components of the device 400 according to an aspect. FIG. 17A illustrates a tool portion according to an aspect. FIG. 17B illustrates a collar component according to an aspect. FIG. 17C illustrates a base component according to an aspect. The device 400 of FIGS. 15-17 may include any of the features previously discussed with reference to FIGS. 1-14.

Referring to FIGS. 15-17, the device 400 may include a shaft 406 operatively coupled to a tool portion including a first moveable arm 408 coupled to a second split portion 409, and a second moveable arm 410 coupled to a first split portion 407. In some examples, the first moveable arm 408 is a component that is integral with the second split portion 409, and the second moveable arm 410 is a component that is integral with the first split portion 407. The first and second split portions 407, 409 may independently roll or rotate with respect to a base 405 such that the first and second moveable arms 408, 410 can move toward and away from each order in order to perform a grasping or cutting operation. In some examples, the base 405 may include one or more features explained with respect to the second curved portion 105 of the previous figures. The base 405 may include a curved portion 484 with a first gear profile 480 configured to contact with a curved surface of the second split portion 409, and a second gear profile 482 configured to contact with a curved surface of the first split portion 407.

The device 400 may include a collar 460 configured to receive at least a portion of the first and second split portion 407, 409, as well as at least a portion of the base 405. The collar 460 may define a first portion 472, a second portion 474, and a connecting portion 476 that connects the first portion 472 and the second portion 474. In some examples, the collar 460 is a unitary component defining the first portion 472, the second portion 474, and the connecting portion 476. In some examples, the first portion 472 may be disposed parallel to the second portion 474. In other examples, the first portion 472 is disposed at an angle with respect to the second portion 474. The space between the first portion 472 and the second portion 474 may define a recess 479. In some examples, the recess 479 may be a U-shaped recess. In some examples, the connecting portion 476 may be a cylindrical portion connected to and disposed between the first portion 472 and the second portion 474. The connecting portion 476 may define an opening 477 configured to receive the base 405. In some examples, the base 405 is inserted into the collar 460 through the opening 477 and the first and second movable arms 408, 410 are inserted into the collar 460 from the recess 479 such that the curved surfaces of the first and second split portions 407, 409 are rollably engaged with the curved portion 484 of the base 405. Also, the opening 477 of the connecting portion 476 may receive activation members 420, which may be coupled to the first movable arm 408 via an opening 464 and coupled to the second movable arm 410 via an opening 462.

The first portion 472, the second portion 474, and the connecting portion 476 may collectively define a U-shape member. However, the collar 460 may have a shape of than a U-shape member. The first portion 472 and the second portion 474 may provide lateral support for the first and second movable arms 408, 410. The connecting portion 476 may define a front edge 481 disposed between the first portion 472 and the second portion 474, and a back edge 483 disposed between the first portion 472 and the second portion 474. The front edge 481 and the back edge 483 may operate as stoppers to prevent the first and second movable arms 408, 410 from further rotation (e.g., prevents the further opening of the cutter or grasper beyond a certain point).

The activation members 420 of the device 400 may include a first activation member 420-1 configured to extend from the shaft 406, through the opening 477 of the collar 460, and extend into and out of the opening 464 defined on the first split portion 407 or the second movable arm 410 such that the first activation member 420-1 extends back towards the shaft 406. Also, the activation members 420 of the device 400 may include a second activation member 420-2 configured to extend from the shaft 406, through the opening 477 of the collar 460, and extend into and out of the opening 462 defined on the second split portion 409 or the first movable arm 408 such that the second activation member 420-2 extends back towards the shaft 406. In some examples, the first and second activation members 420-1, 420-2 may include cables or wires. In some examples, the device 400 may include a first control member 430-1 and a second control member 430-2 configured to adjust a direction of the tool portion in a direction orthogonal to the movement of the first and second movable arms 408, 410. The first and second control members 430-1, 430-2 may be coupled to the collar 460 and an actuator on the shaft 406. In some examples, the first and second control members 430-1, 430-2 include cables or wires.

It is understood that the disclosed embodiments are merely examples, which may be embodied in various forms. Therefore, specific structural and functional details disclosed herein are not to be interpreted as limiting, but merely as a basis for the claims and as a representative basis for teaching one skilled in the art to variously employ the embodiments in virtually any appropriately detailed structure. Further, the terms and phrases used herein are not intended to be limiting, but to provide an understandable description of the embodiments.

It will also be understood that when an element, such as a layer, a region, or a substrate, is referred to as being on, connected to, electrically connected to, coupled to, or electrically coupled to another element, it may be directly on, connected or coupled to the other element, or one or more intervening elements may be present. In contrast, when an element is referred to as being directly on, directly connected to or directly coupled to another element or layer, there are no intervening elements or layers present. Although the terms directly on, directly connected to, or directly coupled to may not be used throughout the detailed description, elements that are shown as being directly on, directly connected or directly coupled can be referred to as such. The claims of the application may be amended to recite exemplary relationships described in the specification or shown in the figures.

The terms “a” or “an,” as used herein, are defined as one or more than one. The term “another,” as used herein, is defined as at least a second or more. The terms “including” and/or “having”, as used herein, are defined as comprising (i.e., open transition). The term “coupled” or “moveably coupled,” as used herein, is defined as connected, although not necessarily directly and mechanically. Accordingly, a singular form may, unless definitely indicating a particular case in terms of the context, include a plural form. Spatially relative terms (e.g., over, above, upper, under, beneath, below, lower, and so forth) are intended to encompass different orientations of the device in use or operation in addition to the orientation depicted in the figures. In some implementations, the relative terms above and below can, respectively, include vertically above and vertically below. In some implementations, the term adjacent can include laterally adjacent to or horizontally adjacent to.

While certain features of the described implementations have been illustrated as described herein, many modifications, substitutions, changes and equivalents will now occur to those skilled in the art. It is, therefore, to be understood that the appended claims are intended to cover all such modifications and changes as fall within the scope of the implementations. It should be understood that they have been presented by way of example only, not limitation, and various changes in form and details may be made. Any portion of the apparatus and/or methods described herein may be combined in any combination, except mutually exclusive combinations. The implementations described herein can include various combinations and/or sub-combinations of the functions, components and/or features of the different implementations described.

What is claimed is:

1. A device comprising:
a shaft;
a tool portion including a first arm and a second arm; and
a split rolling joint including a first curved portion and a second curved portion, the second curved portion being coupled to the shaft, the first curved portion including a first split portion and a second split portion, the first split portion being coupled to the first arm, the second split portion being coupled to the second arm,
at least one of the first split portion and the second split portion being configured to roll with respect to the second curved portion such that at least one of the first arm and the second arm can move towards or away from each other.

2. The device of claim 1, wherein the first split portion and the second split portion are configured to independently roll with respect to the second curved portion.

3. The device of claim 1, wherein each of the first curved portion and the second curved portion includes half a cylinder divided lengthwise.

4. The device of claim 1, wherein each of the first split portion and the second split portion includes a curved surface configured to engage with a surface of the second curved portion.

5. The device of claim 1, wherein each of the second curved portion, the first split portion, and the second split portion includes a gear surface area, the gear surface area including a plurality of recesses and protrusions.

6. The device of claim 5, wherein the gear surface area of the first split portion is rollably engaged with a first portion of the gear surface area of the second curved portion, and the gear surface area of the second split portion is rollably engaged with a second portion of the gear surface area of the second curved portion.

7. The device of claim 5, wherein each of the second curved portion, the first split portion, and the second split portion includes at least one non-geared surface area, the at least one non-geared surface area being devoid of gears.

8. The device of claim 1, further comprising:
an actuation mechanism configured to control movement of at least one of the first split portion and the second split portion.

9. The device of claim 1, wherein the shaft has a diameter between 1 millimeters and 5 millimeters.

10. The device of claim 1, wherein the tool portion is a cutter or a grasper.

11. A device comprising:
a shaft;
a tool portion including a first movable arm and a second movable arm; and
a split rolling joint including a first curved portion and a second curved portion, the second curved portion being coupled to the shaft, the first curved portion including a first split portion and a second split portion, the first split portion being coupled to the first movable arm, the second split portion being coupled to the second movable arm,
the first split portion and the second split portion being configured to independently roll with respect to the second curved portion such that the first movable arm and the second movable arm can move towards or away from each other and can position the tool portion in more than one direction by moving the first and second movable arms as a unit.

12. The device of claim 11, wherein the second split portion is disposed adjacent to the first split portion.

13. The device of claim 11, wherein the second curved portion includes a first row of a gear profile and a second row of the gear profile, the second row being adjacent to the first row, the gear profile including a plurality of recesses and protrusions, the first split portion including a third row of the gear profile, the second split portion including a fourth row of the gear profile,
the third row of the gear profile on the first split portion rollably engaged with the first row of the gear profile on the second curved portion, the fourth row of the gear profile on the second split portion rollably engaged with the second row of the gear profile on the second curved portion.

14. The device of claim 11, further comprising:
a first actuator member coupled to the first movable arm; and
a second actuator member coupled to the second movable arm, wherein movement of the first actuator member is configured to rotate the first split portion about the second curved portion to move the first movable arm, and movement of the second actuator member is configured to rotate the second split portion about the second curved portion to move the second movable arm.

15. The device of claim 14, wherein at least a portion of the first actuator member extends along a longitudinal axis of the shaft, and at least a portion of the second actuator member extends along the longitudinal axis of the shaft.

16. The device of claim 11, wherein the second curved portion includes a guide configured to guide rotation movement of at least one of the first split portion and the second split portion with respect to the second curved portion.

17. The device of claim 11, wherein each of the second curved portion, the first split portion, and the second split portion includes a gear surface area defining a gear profile and at least one non-geared surface area, the at least one non-geared surface area being devoid of gears.

18. A medical device comprising:
a shaft;
a tool portion including a first movable arm and a second movable arm; and
a split rolling joint including a first curved portion and a second curved portion, the second curved portion being coupled to the shaft, the first curved portion including a first split portion and a second split portion, the first split portion being coupled to the first movable arm, the second split portion being coupled to the second movable arm, the second split portion and the second split portion being configured to independently move with respect to the second curved portion,
the first movable arm and the second movable arm being configured to move independently from each other such that movement of the first split portion and the second split portion provides two degrees of movement.

19. The device of claim 18, wherein the two degrees of movement include movement associated with the first split portion rolling on the second curved portion and movement associated with the second split portion rolling on the second curved portion.

20. The device of claim 18, wherein the two degrees of movement include movement associated with moving the first movable arm and the second movable arm towards or away from each other and movement associated with positioning the tool portion in more than one direction by rotating the first and second movable arms as a unit.
